(19) United States
(12) Patent Application Publication
(10) Pub. No.: US 2026/0257007 A1
(43) Pub. Date: Sep. 3, 2026

(54) SYSTEMS, METHODS, AND DEVICES FOR AMBULATORY MEDICAMENT ADMINISTRATION

(71) Applicant: Beta Bionics, Inc., Irvine, CA (US)

(72) Inventors: Mike Mensinger, San Diego, CA (US); Himanshu Patel, Concord, MA (US); Michael J. Rosinko, Peoria, AZ (US); Haripriyan Hampapuran, Carlsbad, CA (US); Sean Saint, San Diego, CA (US)

(73) Assignee: Beta Bionics, Inc., Irvine, CA (US)

(21) Appl. No.: 19/553,950

(22) Filed: Mar. 2, 2026

Related U.S. Application Data
(60) Provisional application No. 63/765,906, filed on Mar. 3, 2025.

(51) Int. Cl. A61M 5/142; A61M 5/172

(52) U.S. Cl. CPC A61M 5/14244; A61M 2005/1726; A61M 2205/50; A61M 2230/201

(57) ABSTRACT

A method for administering medicament from a medical device includes receiving, from a sensor, one or more amounts of biomarkers in blood of a user, determining a response to one or more medicaments responsive to the one or more amounts of biomarkers, adjusting a gain-term based on the response to the one or more medicaments, the gain-term configured to control an amount of medicament that is administered from an ambulatory medical device, and transmitting an instruction to the ambulatory medical device to deliver the amount of medicament to the user.

### FIELD OF THE INVENTION

The present disclosure generally relates to the field of ambulatory medical devices for administering medicaments to patients.

### BACKGROUND

Ambulatory medical devices allow patients the freedom to treat themselves while being mobile. The patient may be tasked with operating the ambulatory medical device. In some cases, the ambulatory medical device is configured to operate without input from the patient. Some ambulatory medical devices are configured to administer medicament responsive to received input from the patient. Many ambulatory medical devices are configured to administer medicament to the patent away from medical supervision. There is a continual need in the art for improvements in ambulatory medical devices to enhance all aspects of their operation.

### SUMMARY

An ambulatory medical device may deliver medicament to a user. The ambulatory medical device determines the amount and rate of medicament to administer over a continuous period of time. The ambulatory medical device may continuously adjust a gain term, which determines the amount of medicament delivered over time.

The gain term may be subject to continuous adjustment based on various parameters and statistics, including biomarkers collected from the user, the user's basal rate, meal times, and data-derived statistics from biomarkers. In some embodiments, the gain term may be adjusted upward or downward based on the user's response to medicament administration.

For example, if a user, who has type 2 diabetes, exhibits a high blood glucose level over a period of time while using the ambulatory medical device, the gain term may be adjusted upward. Conversely, if the user has a low blood glucose level over a period of time, the gain term may be adjusted downward.

### BRIEF DESCRIPTION OF THE DRAWINGS

FIG. 1 is a schematic of an embodiment of the disclosed ambulatory medical device.

FIG. 2 is an illustration showing a user using an embodiment of the disclosed ambulatory medical device.

FIG. 3 is a blown-up view of a system for delivering medicament into a user using an embodiment of the disclosed ambulatory medical device.

FIG. 4 is a blown-up view of an embodiment of the ambulatory medical device for delivering medicament into a user.

FIG. 5 is a schematic of an embodiment of a system for adjusting an insulin sensitivity factor in an embodiment of the ambulatory medical device.

FIG. 6 is an illustration of a screen that a user may interact with in an embodiment of the disclosed ambulatory medical device.

FIG. 7A is a set of screenshots showing a graph screen for a continuous glucose monitor.

FIG. 7B is a set of screenshots showing a meal announcement interface.

FIG. 8 is a flow diagram of a system for adjusting an insulin sensitivity factor in an embodiment of the disclosed ambulatory medical device.

FIG. 9 is a schematic of a computer system that may perform processing tasks or other computing tasks in various embodiments of the disclosed ambulatory medical device.

### DETAILED DESCRIPTION

The disclosed subject matter is a system for delivering medicaments using an ambulatory medical device. The system determines the amount of medicament to deliver to a user based on input and feedback from the user. In various embodiments, the system may determine an amount of medicament based on biomarker feedback from the user. For example, various algorithms may be used to determine the amount of medicament to deliver to the user. The amount of medicament may be varied continuously and dynamically over time.

In an exemplary embodiment, the medicament may include insulin. In another exemplary embodiment, the medicament may include glucagon. Insulin is a hormone that regulates blood glucose levels by facilitating glucose uptake into cells. Glucagon is a hormone that raises blood glucose levels by promoting the release of stored glucose from the liver. In various embodiments, the medicament may be delivered automatically by the ambulatory medical device. The term ambulatory, as used herein, describes a device that may be worn by the user and is not connected to any stationary device such as connected to a wall or outlet. The ambulatory medical device allows the user to freely travel and go where they please while the ambulatory medical device treats the patient or user.

In various embodiments, the ambulatory medical device may include an insulin sensitivity factor. The insulin sensitivity factor may be determined using various algorithms and may be adjusted continuously over time. The insulin sensitivity factor, as used herein, may include a gain term that determines the aggressiveness with which the patient is treated with insulin. In various embodiments, the gain term may also be applied to other medicaments besides insulin. In an exemplary embodiment, the ambulatory medical device may determine an amount of unmetabolized insulin in the user. In yet another exemplary embodiment, the ambulatory medical device may determine a basal rate in the user. The basal rate, as used herein, may refer to the rate at which insulin is produced by the user. In yet another exemplary embodiment, the ambulatory medical device may include one or more meal adaptations. For example, the ambulatory medical device may learn to adjust medicament delivery based on meals taken by the user.

The ambulatory medical device may connect to a user to deliver medicament to the user. For example, the ambulatory medical device may be worn by the user administer medicament using a transdermal needle. The ambulatory medical device may include a pump that delivers medicament to the user. An example of a pump may be a peristaltic pump, which moves fluid through a flexible tube using rotating rollers. The ambulatory medical device may further include a controller that determines an amount of medicament and instructs the pump to deliver the medicament to the user. The ambulatory medical device may also include one or more reservoirs for medicament. The controller may determine an amount of medicament to deliver to the user. The ambulatory medical device may continuously modify or adjust the amount of medicament based on time and various sensor inputs from the user. For example, the ambulatory medical device may collect biomarker data from the user and adjust the amount of medicament to be delivered. The ambulatory medical device may also be operated by the user, such as allowing the user to enter meal times or meal breaks and specify the size of a meal.

An exemplary embodiment of the ambulatory medical device includes a corrections controller. The corrections controller may be a control mechanism that calculates small insulin doses based on near-continuous calculations of the deviation of current glucose from a target level. The controller may include a gain term that controls how many units of insulin to deliver per mg/dL that the glucose is above target. The gain term can be considered an insulin sensitivity factor or an aggressiveness term. Additionally, the controller may take into account any insulin that has been delivered but has not yet been metabolized. The insulin amount that has not yet been metabolized is the insulin on board.

The gain term of the ambulatory medical device controls an amount of medicament delivery. For example, the gain term in an insulin-delivering medical device may adjust the insulin sensitivity factor, which determines the amount of medicament delivered per time period to a user. A higher gain term may result in more aggressive medicament delivery, while a lower gain term may result in less aggressive medicament delivery. The gain term may change over time. The ambulatory medical device may collect biomarker data and adapt the gain term based on the biomarker data. For example, the gain term may be modified by the ambulatory medical device based on glucose readings from the user. The ambulatory medical device may vary the gain term based on each user. Further, the gain term may be adjusted based on past delivery data.

The gain term may be initialized based on various parameters such as body mass, total daily dose, and similar factors. In an exemplary embodiment, the gain term may be adapted based on glucose statistics. For example, a target glucose statistic may be determined by evaluating glucose levels at regular time intervals. If glucose at the end of a time period is greater than a conditional threshold, the gain term may be increased by a set percentage. If glucose at the end of the time period is below a conditional threshold, the gain term may be decreased by a set percentage.

Similarly, the gain term may be adjusted based on mean glucose levels. If the mean glucose is greater than a target, such as 150 mg/dL, the gain term may be increased. If the mean glucose is less than the target, the gain term may be decreased. Other statistical methods may also be used to adjust the gain term. For example, both the mean and standard deviation of glucose levels may be used to calculate a lower glucose limit. The lower limit may be determined using the measured mean, standard deviation, and a t-statistic with a significance level of 0.10. If the calculated lower statistic is below a threshold, the gain term may be decreased. If the calculated lower statistic is above the threshold, the gain term may be increased.

The gain term may also be adjusted based on the statistics of medicament delivery. For example, a patient may have a specific total daily insulin dose that is expected, and the gain term may be increased if the actual insulin delivered is below this target. Similarly, in a bi-hormonal system using insulin and glucagon, the insulin gain term may be decreased when glucagon dosing is higher than expected.

In an exemplary embodiment, the ambulatory medical device may determine insulin on board and adapt various medicament delivery amounts based on the insulin on board. The insulin on board, as used herein, refers to unmetabolized insulin in the patient. Every patient or user metabolizes insulin at a different rate. Accordingly, the ambulatory medical device may adjust the determination of insulin on board specifically for each user. A determination of the remaining un-metabolized insulin in the patient may be performed using an estimate of insulin t-max, which is the time at which the pharmacodynamic effect of a single insulin dose reaches its maximum level. Alternatively, the calculation may use insulin duration, which represents the total period during which a single dose of insulin affects the patient's glucose levels. These insulin activity parameters may vary from patient to patient. The system may adapt these parameters to the individual patient.

The ambulatory medical device may also factor in a basal rate for the user. Each patient may have a basal rate of endogenous glucose production, which ideally may be compensated for by the basal insulin rate. The nominal basal rate can be estimated and adapted, with instantaneous adjustments made based on glucose levels. For example, a sensor may receive glucose readings from the user and transmit them to the controller. Based on the glucose levels, a basal rate can be estimated. In various embodiments, an initial basal rate may be estimated based on user parameters such as body mass and total daily dose.

In an exemplary embodiment, a nominal basal rate may be adjusted using a method similar to the insulin sensitivity factor adaptation referenced above. For example, glucose levels may be evaluated over a 24-hour period, with readings collected every six hours. Each time a glucose level exceeds a threshold, the basal rate may be decreased by a certain percentage. Each time a glucose level falls below the threshold, the basal rate may be increased by a certain percentage.

In an exemplary embodiment, the basal rate may be determined based on the amount of medicament previously delivered. For example, a nominal basal rate may represent the amount of insulin required to counteract a patient's nominal endogenous glucose production (EGP). The EGP rate may correspond to the lowest amount of insulin delivered throughout the day. Accordingly, the nominal basal rate may be adjusted by monitoring insulin delivery over time. For instance, during a time period with the lowest insulin delivery, the basal rate may be adjusted based on changes in insulin on board and glucose levels, setting the basal rate to the nominal basal rate.

The adjustments for insulin on board (IOB) may be made by subtracting the IOB at the beginning of the period from the IOB at the end of the period and using this difference to adjust the measured insulin delivery. Similarly, if glucose levels dropped during the period, the measured insulin delivery may be decreased, as it indicates that insulin delivery exceeded the patient's endogenous glucose production. Conversely, if glucose levels increased, the measured insulin delivery may be increased.

In an exemplary embodiment, the ambulatory medical device includes a Meal Controller. The Meal Controller may adjust medicament delivery based on meals taken by the user. For example, a user may use one or more inputs on the ambulatory medical device to indicate that a meal is being taken. The ambulatory medical device may then adjust medicament delivery based on this input. In various embodiments, the ambulatory medical device may learn from past meals and biomarker feedback to adjust the amount of medicament or insulin delivered in response to a meal. For example, if a user exhibits a consistent biomarker reaction to a specific meal over time, the ambulatory medical device may adjust insulin delivery near mealtime based on the user's historical biomarker data.

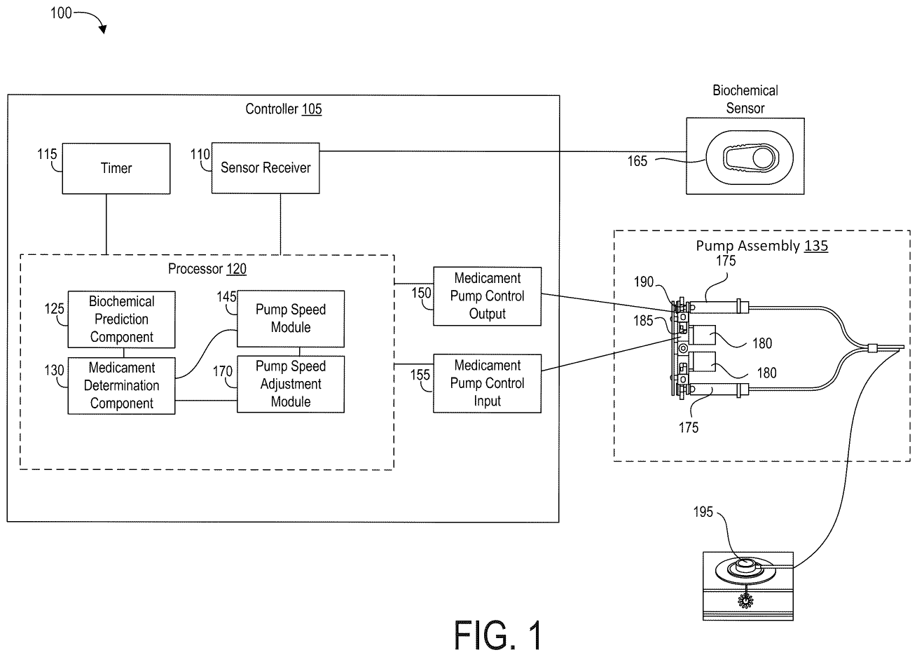

Referring to FIG. 1, FIG. 1 is a schematic 100 of an embodiment of the disclosed ambulatory medical device. The ambulatory medical device may be worn by a user, allowing the user to move freely while the ambulatory medical device delivers a medicament at a prescribed rate. The ambulatory medical device may collect biomarker data from the user to adjust medicament delivery. The ambulatory medical device may be configured to deliver one or more medicaments based on the user's specific parameters. For instance, if the user has type 2 diabetes, an ambulatory medical device can adjust insulin delivery variably based on activity level, meal times, basal rate, and past medicaments. Accordingly, the ambulatory medical device may continuously adjust the amount of medicament delivered to the user based on a variety of parameters.

In the exemplary embodiment of the ambulatory medical device shown in schematic 100, the ambulatory medical device includes a controller 105, a biochemical sensor 165, a pump assembly 135, and a transdermal syringe 195. The controller 105 may collect various data, such as user input, biomarker sensor data from a biochemical sensor 165, and other information, including time and wireless communication data from one or more sources. The biochemical sensor 165 may include one or more sensors that collect biomarker data from the user. For example, the biochemical sensor 165 may include a glucose sensor attached to the user. An example of a glucose sensor may be a continuous glucose monitor (CGM) that measures interstitial glucose levels in real time. The pump assembly 135 may include one or more reservoirs, pumps, and motors configured to deliver medicaments from the reservoirs to the user in response to instructions from the controller 105. The transdermal syringe 195 may attach to the user and provide a connection that allows medicament to be delivered from the medicament pump assembly to the user.

The controller 105 may control medicament delivery to the user and determine modifications in medicament delivery. The controller 105 may receive input from various sources, such as the user, the biochemical sensor 165, and other relevant data sources. The controller 105 may adjust medicament delivery based on a variety of factors, including learning over periods of time from a user's historical readings, such as historical glucose readings. In the exemplary embodiment of the controller 105 shown in schematic 100, the controller 105 may include a timer 115, a sensor receiver 110, a processor 120, a medicament pump control output 150, and a medicament pump control input 155.

The sensor receiver 110 may receive various data from outside the ambulatory medical device. For example, the sensor receiver 110 may receive data from a biochemical sensor 165. Examples of data received from the biochemical sensor 165 may include biomarker data such as blood glucose levels, blood oxygen levels, body temperature, and similar physiological metrics. The sensor receiver 110 may also receive data from other sources. For example, the sensor receiver 110 may receive signals from outside the ambulatory medical device to initiate or modify certain actions. An example of a sensor receiver 110 receiving external signals may include retrieving historical user data from a database server. In another example, the sensor receiver 110 may receive an instruction from a server to perform a specific action. In one instance, the sensor receiver 110 may start or stop a treatment based on an instruction received from a server.

In various embodiments, the sensor receiver 110 may be wired or wireless. Sensor receiver 110 may connect to one or more biochemical sensors or other sensors collecting biomarker information from the user. In various embodiments, the sensor receiver 110 may include both wired and wireless connections to various sensors on the user, as well as collect data or instructions from external sources. In some embodiments, sensor receiver 110 may be configured to send signals as well as receive them. For example, sensor receiver 110 may be an antenna configured to transmit data from the ambulatory medical device to a server. For instance, the ambulatory medical device may be configured to transmit medical information to a server used by a medical practitioner to evaluate the user.

In one example, the sensor receiver 110 is an antenna that is capable of sending and receiving data. The antenna in the ambulatory medical device may use Bluetooth to send and receive data from other devices. The ambulatory medical device may use other forms of wireless communication, including but not limited to Wi-Fi, LTE, and NFC. In various embodiments, the wireless communication antenna that transmits and receives wireless data is placed as far as practicable from components and materials that may interfere with the wireless signal within the ambulatory medical device. In one embodiment, the ambulatory medical device comprises a metal housing connected to a non-conductive bezel. The wireless communication antenna is placed adjacent to the non-conductive bezel such that interference with the wireless signal from the metal housing is diminished compared to other locations inside the ambulatory medical device.

The timer 115 on the ambulatory medical device may track various aspects of medicament treatment. For example, the timer 115 or the processor 120 may collect biomarker statistics from the user. In one instance, the processor 120 may collect blood glucose levels from the user every six hours. In another instance, the processor 120 may calculate an average glucose level over a six-hour period. The timer 115 may be used to schedule the collection of biomarker events in various embodiments. The timer 115 may also be used to regulate the timing of medicament delivery. For instance, the processor 120 may issue an instruction for the pump to deliver a specified amount of medicament over a designated time period. The timer 115 may be utilized to deliver medicament at an appropriate rate. In various embodiments, the processor 120 may instruct the pump to continuously update the rate of medicament delivery over time. Accordingly, the timer 115 may be used to determine how the rate changes. In various embodiments, the processor 120 may adjust a gain term based on statistics collected using the timer 115. Additionally, the processor 120 may adjust a gain term based on an anticipated mealtime, which may also be determined partially by the timer 115.

The medicament pump control output 150 may receive instructions from the processor 120 to control the output of the medicament pump 175. For example, the processor 120 may determine a rate to administer medicament and transmit an instruction to the medicament pump control output 150 to deliver medicament at that rate. The medicament pump control output 150 may then transmit the instruction to medicament pump assembly via an electric signal to pump at the desired rate. In various embodiments, the processor 120 may transmit an instruction to deliver a specific volume of medicament to the user. The medicament pump control output 150 may transmit an instruction to deliver the volume of medicament at a default rate. The medicament pump control output 150 may further transmit an instruction to stop medicament delivery once the specified volume has been administered.

The medicament pump control output 150 and the medicament pump control input 155 may send and receive data to and from the pump assembly 135, respectively. The pump assembly 135 may be connected to the controller 105 via a wired or wireless connection. If the pump assembly 135 is connected via a wireless connection, it may include a transceiver capable of sending and receiving signals to and from the controller 105.

The medicament pump control input 155 may receive signals from the pump assembly 135. In an embodiment, the medicament pump control input 155 may receive a signal from the pump assembly 135 that tabulates the amount or volume of medicament delivered to the user. Accordingly, data collected by the medicament pump control input 155 may account for any differences between the desired volume of medicament to be administered and the actual amount administered. In an exemplary embodiment, a magnetic encoder on the pump assembly 135 may record the number of times gears spin within the pump assembly 135, where the gears control the amount of medicament delivered. The medicament pump control input 155 may transmit data from the magnetic encoder to the processor 120 to determine the actual amount of medicament administered. The processor 120 can adjust the rate or volume of medication administered based on data from the medicament pump control input 155.

The processor 120 may collect data from outside the ambulatory medical device, control various functions of the ambulatory medical device, determine treatment administered by the ambulatory medical device, and communicate with or send instructions to other devices. The processor 120 may be a computing system capable of performing various computing tasks, such as determining an amount of medicament to be administered to a user, receiving data and instructions from outside the ambulatory medical device, and similar operations. In the embodiment of the processor 120 shown in the schematic 100, the processor 120 may include a biochemical prediction component 125, a medicament determination component 130, a pump speed module 145, and a pump speed adjustment module 170.

The biochemical prediction component 125 may determine or predict various biological processes in the user. For example, the biochemical prediction component 125 may be configured to determine an insulin t-max level in the user. The t-max, as used herein, is the time at which the insulin level, or other chemical, reaches its maximum in the user. The biochemical prediction component 125 may also be configured to determine a basal rate in the user. For example, the biochemical prediction component 125 may predict the rate at which insulin is produced in the user at various times of the day. For instance, the biochemical prediction component 125 may predict a basal rate in the morning, afternoon, and evening for a user. The biochemical prediction component 125 may further determine various medical processes occurring in the user based on data collected by the controller 105. For example, if the biochemical sensor 165 sends data indicating an increased temperature in the user, the biochemical prediction component 125 may predict that the user's metabolic rate is increasing and respond by adjusting medicament delivery.

The medicament determination component 130 may determine an amount of medicament or rate of medicament delivery to administer treatment to the user. For example, the medicament determination component 130 may determine a default rate to administer to the user. In some embodiments, the medicament determination component 130 may use one or more biomarker sensor readings collected from the biochemical sensor 165 to adjust the medicament treatment rate. For example, if the biochemical sensor 165 collects data indicating that the user's blood glucose level is high, the medicament determination component 130 may increase the rate of medicament delivery to address the elevated glucose level. For instance, the medicament determination component 130 may increase the rate of insulin delivered to the user. In some embodiments, the medicament determination component 130 may determine a dynamic rate of medicament delivery that continuously changes over time. For example, a user may have a historical reaction to meals. In one instance, data collected over a long period of time may indicate a blood glucose response to a lunch meal. Accordingly, the medicament determination component 130 may determine an insulin delivery rate that continuously adjusts for a period of time after a meal based on historical data.

In further examples, the medicament determination component 130 may adjust a medicament delivery rate based on the t-max values determined by biochemical prediction component 125. For instance, medicament determination component 130 may lower the medicament delivery rate at t-max and gradually increase the medicament delivery rate afterward. The medicament determination component 130 may adjust the medicament delivery rate before and after t-max periods. In another example, the medicament determination component 130 may adjust medicament delivery based on the basal rate determined by the biochemical prediction component 125. For example, the biochemical prediction component 125 may determine a basal rate of insulin production in the body of the user. The medicament determination component 130 may subtract the predicted insulin production, as determined by the basal rate, from the calculated medicament delivery rate.

The pump speed module 145 in the processor 120 converts the output from the medicament determination component 130 into a pump speed that delivers medicament at the rate and amount determined by the medicament determination component 130. For example, the medicament determination component 130 may specify a rate of medicament administration. The pump speed module 145 may convert that rate into a pump speed and transmit a signal to a motor that operates the pump. The pump speed module 145 may determine an instruction that causes the motor to spin and operate the pump at a set speed. In some embodiments, the medicament determination component 130 may determine a volume of medicament to administer to a user. Accordingly, the pump speed module 145 may send a signal that operates the pump for a specified time to deliver the determined volume of medicament. In some embodiments, the medicament determination component 130 may determine a rate of medicament administration over a period of time where the rate changes dynamically. The pump speed module 145 may send an instruction that causes the pump to administer medicament at a dynamically changing rate over time.

The pump speed adjustment module 170 may adjust the speed of the pump speed module 145 based on various data. For instance, data collected via the medicament pump control input 155 may indicate a difference between the actual medicament delivered and the determined medicament delivery. Accordingly, the pump speed adjustment module 170 may adjust the rate of medicament delivery based on this difference.

The biochemical sensor 165 may be a sensor that collects various biomarker data from the user. For example, the biochemical sensor 165 may collect blood glucose levels in the user. An example of a blood glucose sensor is a continuous glucose monitor (CGM), such as the Dexcom G7 or the FreeStyle Libre, which continuously measures interstitial glucose levels and transmits data in real time. Other biochemical sensors that may be used for diabetic patients include ketone sensors, which monitor blood ketone levels to detect diabetic ketoacidosis (DKA), and lactate sensors, which measure blood lactate levels as an indicator of metabolic stress. The biochemical sensor 165 may transmit sensor data to the controller 105.

In some embodiments, the biochemical sensor 165 may transmit data through a wired connection. In other embodiments, the biochemical sensor 165 may transmit data wirelessly. In some embodiments, the biochemical sensor 165 may also receive data instructions. For example, the biochemical sensor 165 may modify one or more parameters in sensor collection based on an instruction received from the controller 105. For instance, the controller 105 may instruct the biochemical sensor 165 to collect data from additional biomarkers. As an example, the controller 105 may instruct the biochemical sensor 165 to collect body temperature data in addition to blood glucose levels. The controller 105 could also direct the biochemical sensor 165 to save energy by collecting biomarker data less frequently.

The pump assembly 135 is an apparatus capable of holding medicament and pumping it to administer the medicament to the user. The pump assembly 135 may include reservoirs, pumps, motors, and connections between the motors and pumps to operate the pumps. In the embodiment of the pump assembly 135 shown in schematic 100, the pump assembly 135 includes a medicament pump 175, DC motors 180, gears 185 connecting the DC motors 180 to the medicament pump 175, and magnetic encoders 190 to record the amount of medicament administered.

The pump assembly 135 may be connected to the user via a transdermal syringe 195. The transdermal syringe 195 may be configured to inject medicament into fatty tissue below the skin of the user. In some embodiments, the pump assembly 135 may pump a medicament through one or more tubes to the transdermal syringe 195. In embodiments where the pump assembly 135 carries more than one type of medicament, it may be connected to multiple transdermal syringes.

In an example of use, the processor 120 may transmit an instruction to the pump assembly 135 to deliver insulin at a specified rate to the user. In another example, the pump assembly 135 may pump insulin at the instructed rate through tubing to the transdermal syringe 195, where the medicament enters the user. In some embodiments, the pump assembly 135 may be connected to the user in alternate ways. For example, in addition to the transdermal syringe 195, the pump assembly 135 may administer medicament through other delivery methods.

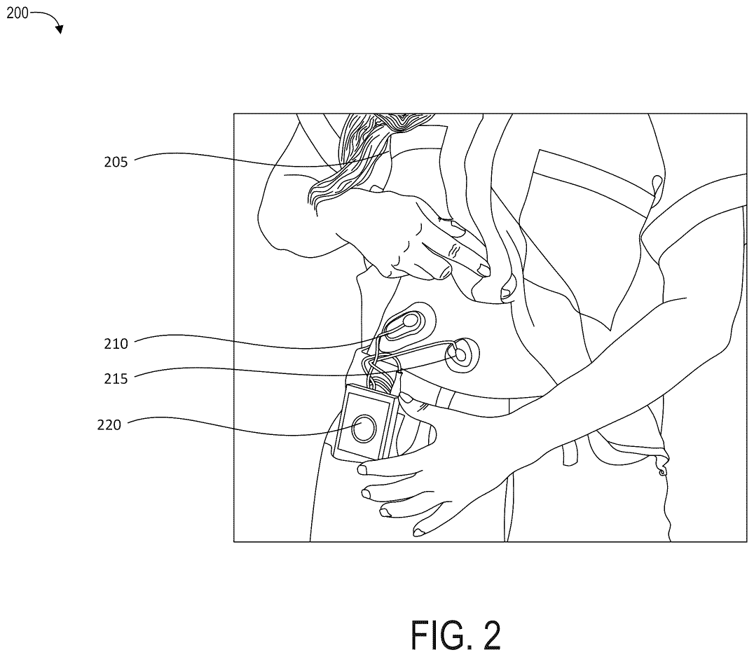

Referring to FIG. 2, FIG. 2 is an illustration 200 showing the user 205 using an embodiment of the disclosed ambulatory medical device 220. The ambulatory medical device 220 may attach to the user 205, allowing the user 205 to walk freely and move to various locations, including outside a medical setting. The ambulatory medical device 220 may be operated by the user 205 and/or by a medical practitioner.

In an example of use, a medical practitioner may configure ambulatory medical device 220 with initial parameters, and the user 205 may operate the ambulatory medical device 220 thereafter. The ambulatory medical device 220 may include one or more input options. Further, the ambulatory medical device 220 may include a processor, a medicament, and a means to administer the medicament to a user 205. In some embodiments, the ambulatory medical device 220 may include a biochemical sensor 210. The biochemical sensor 210 may attach to a user 205 at various locations. As shown in the illustration 200, the biochemical sensor 210 is attached to the user 205 at the side of the abdomen.

The ambulatory medical device 220 may be configured to administer medicaments through a transdermal syringe 215, which is attached to the user 205. Accordingly, the medicament in the transdermal device may be pumped through tubing and delivered into the user 205 via the transdermal syringe 215.

The ambulatory medical device 220 may include various options for the user 205 to input data or operate the ambulatory medical device 220. For example, the ambulatory medical device 220 may include a touchscreen display that enables the user 205 to modify settings or operate the ambulatory medical device 220. For instance, the user 205 may inform the ambulatory medical device 220 when they are about to have a meal. Additionally, the user 205 may provide information regarding the size of the meal. For example, the user 205 may input whether the meal is small, medium, or large. The ambulatory medical device 220 may adjust medicament delivery based on parameters and input from the user 205.

The ambulatory medical device 220 may also learn over time based on data collected from the biochemical sensor 210 and adjust medicament delivery accordingly. For example, the ambulatory medical device 220 may be configured to collect blood glucose levels from the user 205 at various times of day and, based on these levels, determine the basal rate of insulin production in the user 205. The ambulatory medical device 220 may then adjust the insulin delivery rate throughout the day based on learned and collected data.

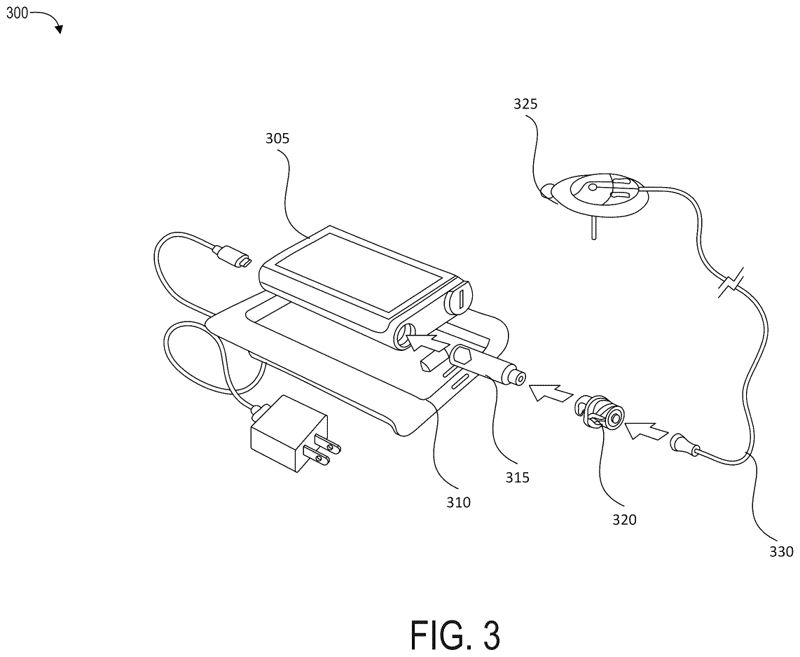

Referring to FIG. 3, FIG. 3 is a blown-up view of a system 300 for infusing medicament into a user using an embodiment of the disclosed ambulatory medical device 220. The system 300 may include various components, including a controller 305, a cartridge 315, a connection adapter 320, flexible tubing 330, and an infusion set 325. In certain embodiments, the controller 305 may be designed to be positioned in a base 310 when it is not being utilized by the user 205. The cartridge 315 may be configured to hold various medicaments in liquid form. In some embodiments, the ambulatory medical device 220 may be charged via a wall charger that connects to it.

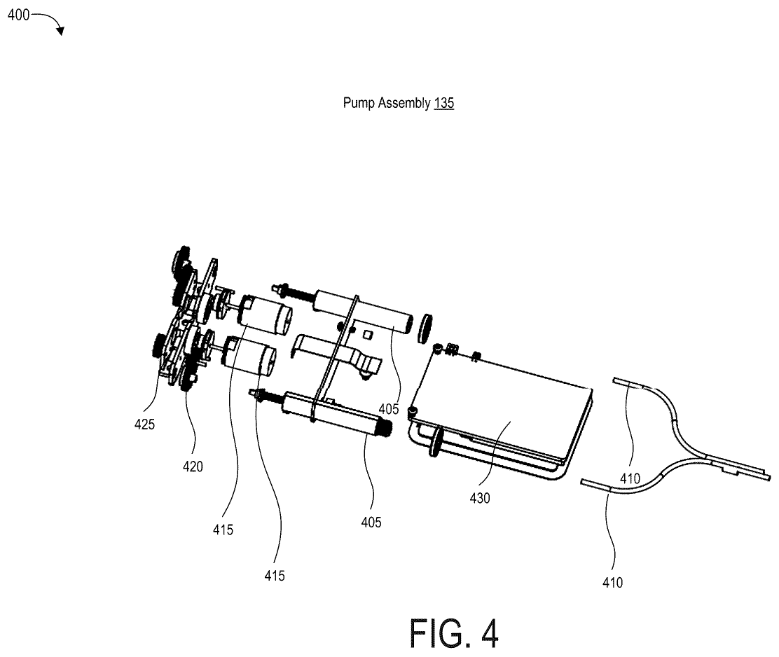

Referring to FIG. 4, FIG. 4 is a blown-up view 400 of an embodiment of the ambulatory medical device 220 for infusing medicament into a user. The ambulatory medical device 220 may comprise a variety of designs and components. The embodiment shown in the view 400 includes a controller 430, one or more cartridges 405, where the cartridges 405 include a pump, and one or more motors 415, where the motors 415 are capable of spinning gears 420. Additionally, the view 400 includes a magnetic encoder 425, which records the spinning of gears 420 to determine the amount of medicament that was pumped.

In some embodiments, the gears 420 may be connected to the cartridges 405, such that spinning the gears 420 operates the pump in the cartridges 405. When the pump is activated, the liquid held in the cartridges 405 may be pumped through tubing 410 and delivered to the user 205.

As shown in the view 400, the embodiment of the ambulatory medical device 220 includes two cartridges 405. In some embodiments, the cartridges 405 may hold different types of medicaments. In other embodiments, the cartridges 405 may hold the same type of medicament. Each cartridge 405 is connected to a separate tubing 410, which delivers medicament to the user 205. Instructions from the controller 430 may cause the motors 415 to spin, which in turn causes the gears 420 to spin, operating the pumps and delivering medicament from the cartridges 405 through the tubing 410 and into the user 205.

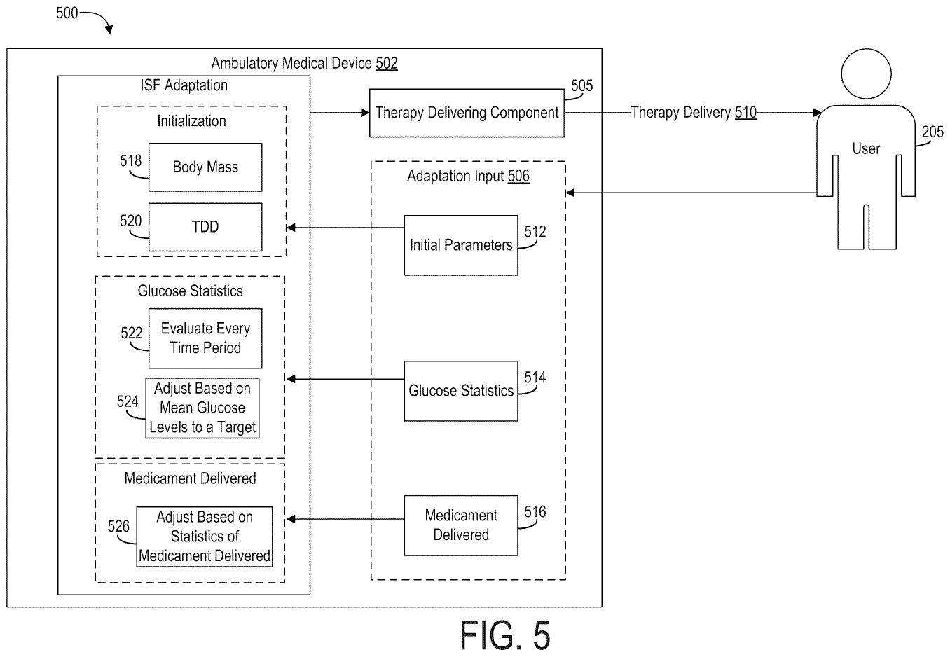

Referring to FIG. 5, FIG. 5 is a schematic 500 of an embodiment of a system for adjusting an insulin sensitivity factor in an embodiment of the ambulatory medical device 220. The insulin sensitivity factor, as used herein, will be referred to as the gain term. The gain term may specify the amount of insulin or medicament delivered per step from the medical device 220.

A step may be a unit of time or another defined interval that represents a segment of time. The ambulatory medical device 220 can deliver a volume of medication over time in multiple steps. The gain term may determine the amount of medicament delivered per step and, accordingly, per unit of time.

The gain term may be determined in the ISF adaptation box in the schematic 500 based on an adaptation input 506. Some examples of the adaptation input 506, as shown in the schematic 500, include initial parameters 512, glucose statistics 514, and medicament delivered 516.

The adaptation input 506 may be collected as initial parameters and input by medical practitioners. Additionally, the adaptation input 506 may be entered directly and explicitly by the user 205. In some embodiments, the adaptation input 506 may be collected by one or more biochemical sensors 210 attached to the user 205. The adaptation input 506 may also be derived from a combination of these sources. Further, the adaptation input 506 may be retrieved from historical data stored in a database, either on or off the ambulatory medical device 502.

Examples of the initialization parameters 512 include body mass 518 and total daily dose 520. These parameters may be input directly by a medical practitioner or by the user 205. Some initialization parameters 512 may also be collected by a sensor, such as initial biomarkers like blood glucose readings or other biomarker data. The glucose statistics 514 may include various statistics derived from biochemical or biomarker readings of the user 205's blood glucose levels. For example, blood glucose statistics determined from an evaluation every time period 522 may be used to adjust the gain term.

In glucose statistics, blood glucose levels are measured at regular intervals for each period 522. The time period could be six hours. At the end of six hours, the gain term is adjusted based on the blood glucose measurement.

For example, if the blood glucose measurement is below a target value, the gain term is lowered by a percentage. If blood glucose exceeds the target value at the period's end, the gain term increases by a set percentage. Once the gain term is adjusted, the amount of insulin delivered per step is directly proportional to the gain term.

In another example of a glucose statistic, the gain term may be adjusted based on mean glucose levels to a target 524. For example, the average glucose level can be measured over 24 hours. At the end of the time period, the gain term is adjusted based on whether the mean glucose level is above or below a target value.

For example, if the mean glucose level is above the target value, the gain term may be adjusted upward. If the mean glucose level is below the target value at the end of the time period, the gain term may be lowered by a set percentage. The set percentage and target value may be adjusted based on the user 205. An example of a target value may be 100 milligrams per deciliter of glucose.

In another embodiment, another glucose statistic that may be measured is standard deviation. Standard deviation quantifies the amount of variation or dispersion in glucose levels from the mean over a period of time. A higher standard deviation indicates greater fluctuations in glucose levels, while a lower standard deviation indicates more stable glucose levels.

A combination of the mean and standard deviation may also be used to adjust the gain term. For example, if the standard deviation is high, indicating significant glucose fluctuations, the gain term may be adjusted to compensate for variability. If the standard deviation is low, the gain term may remain stable or be adjusted downward.

In yet another embodiment, the gain term may be adjusted by the medicament delivered 516. For example, the gain term may be adjusted based on statistics of the medicament delivered 526. If the user 205 has a total daily dose, a target amount of medicament can be determined for any given time of day. If the actual amount of medicament delivered falls below a target threshold for that time, the gain term may be adjusted based on the amount of medicament already delivered to the user 205.

In another example, where both insulin and glucagon are administered to the user 205, the gain term for insulin may be adjusted based on the amount of glucagon delivered. For instance, the gain term for insulin may be adjusted downward when the amount of glucagon administered to the user 205 exceeds a threshold.

The processor 120 uses the gain term to determine how much medicament to deliver to the user 205 over time. The medicament may be determined by the medicament determination component 130, whereby an instruction is passed to the therapy delivering component 505, such as the pump assembly 135, which administers the medicament to the user 205.

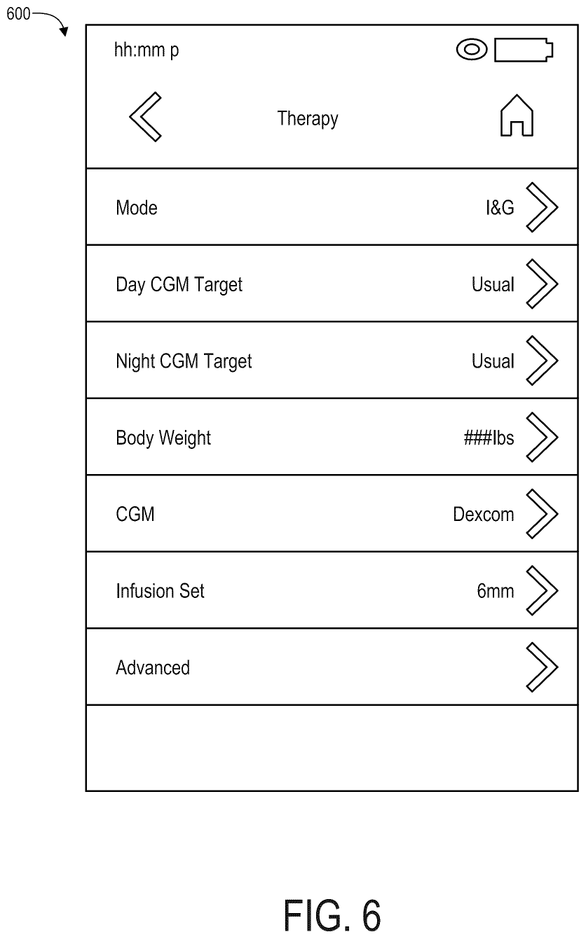

Referring to FIG. 6, FIG. 6 is an illustration of a screen 600 that a user may interact with in an embodiment of the disclosed ambulatory medical device 220. The ambulatory medical device 220 may include multiple input options for the user 205. These input options may allow the user 205 to modify various parameters that affect the determination of medicament delivery, the amount of medicament administered, and the timing of medicament delivery.

Some parameters may affect the determination of the gain term for the user 205. Other parameters may influence when medicament is delivered and how much variability exists in delivery over a given time period. Examples of inputs that the user 205 may enter into the ambulatory medical device 220 include day and night continuous glucose monitor targets. For example, the user 205 may enter a daytime glucose target of 100-130 mg/dL and a nighttime glucose target of 90-120 mg/dL.

Additionally, the user 205 may input their body weight, which may affect the initial gain term. Other inputs may be related to hardware settings, such as the mode selection, which determines the types of hormones used for treatment. In the mode shown in the screen 600, the ambulatory medical device 220 is configured to operate with both insulin and glucagon.

The user 205 may modify the mode selection, which may result in an adjustment of the gain term. For example, a mode using only insulin may have a higher gain term than a mode using both insulin and glucagon.

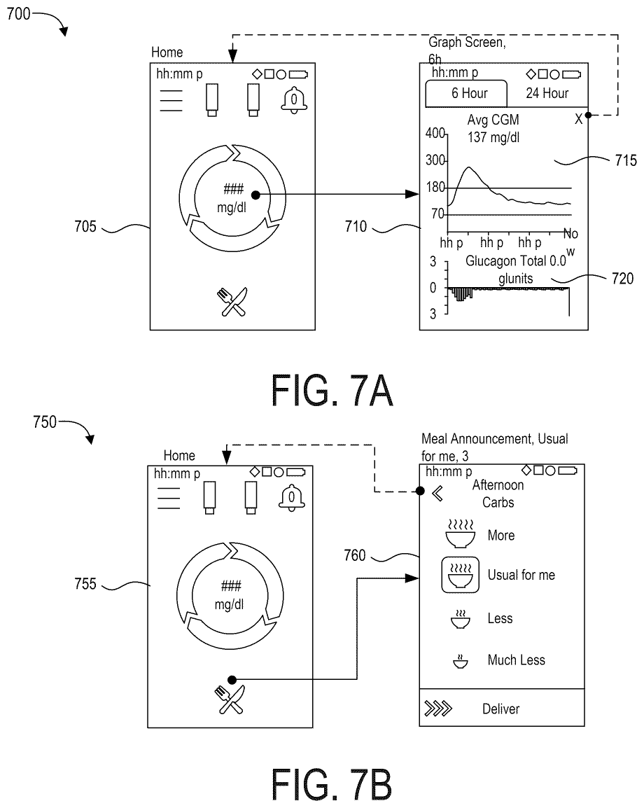

Referring to FIG. 7A, FIG. 7A is a set of screenshots 700 showing a graph screen for a continuous glucose monitor. A first screenshot 705 displays a user input screen, allowing the user 205 to select various options to operate the ambulatory medical device 220. A selection in the center of the first screenshot 705 may navigate the user 205 to a graph screen 710. The graph screen 710 displays various statistics related to the user 205's treatment with the ambulatory medical device 220.

The upper graph 715 displays an average continuous glucose monitor reading over a time period for the user 205. Continuous glucose monitor readings track glucose levels for the user 205 over a six-hour period. As shown in the graph 715, the user 205's glucose level spiked early in the time period, rising above a threshold range, and then returned within the threshold range for the majority of the time period. Accordingly, the average glucose level for the time period falls within the threshold range shown in the graph 715. However, the standard deviation may indicate that the spike in glucose level could result in an increase in the gain term.

The lower graph 720 represents the amount of glucagon administered to the user 205. The lower graph 720 shows that early glucagon administration caused a spike in glucose levels, as seen in the upper graph 715. The amount of glucagon administered to user 205 may be used by the processor 120 to adjust the gain term and determine the appropriate amount of insulin to be delivered.

Referring to FIG. 7B, FIG. 7B is a set of screenshots 750 showing a meal announcement interface. The ambulatory medical device 220 may include a meal announcement interface that allows the user 205 to announce a meal. The meal announcement may prompt ambulatory medical device 220 to modify or adjust the medicament administration rate in anticipation of the meal.

A first screen 755 presents the user 205 with a set of icons or buttons on a touchscreen display. An icon at the bottom of the first screen 755 allows the user 205 to select a meal announcement. A second screen 760 displays the meal announcement interface, where the user 205 can select a meal size. The selected meal size may be used to adjust the gain term.

A larger meal may result in an upward adjustment of the gain term, whereas a small meal may result in a lowering of the gain term by a percentage or set amount. Likewise, a medium-sized meal may result in little to no change in the gain term.

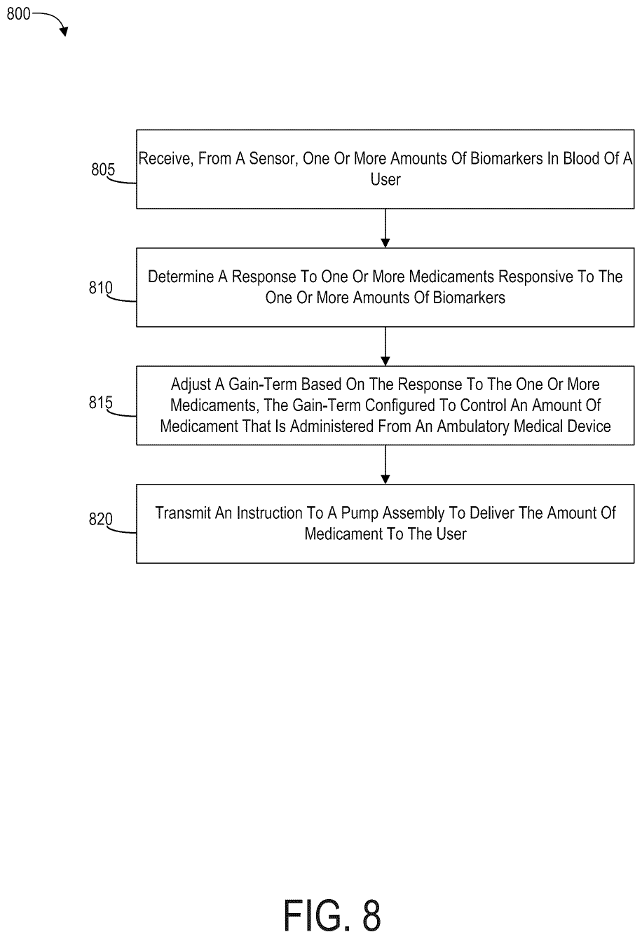

Referring to FIG. 8, FIG. 8 is a flow diagram 800 of a system for adjusting an insulin sensitivity factor in an embodiment of the disclosed ambulatory medical device 220. The insulin sensitivity factor, which is also referred to herein as the gain term, determines the amount of insulin administered to the user 205 over a period of time. A higher gain term results in more insulin being administered, whereas a lower gain term results in less insulin being administered.

The gain term may be adjusted based on various parameters received by the ambulatory medical device 220 in different embodiments. It may also be adjusted based on learned parameters, such as the amount of insulin needed for a meal or the insulin requirements of the user 205 at different times of day. A calculation for t-max of insulin for the user 205 may be factored into the gain term.

The controller 105 may calculate t-max for insulin from the user's historical glucose data. The t-max for insulin represents the time after administration when the insulin reaches its peak effect. The basal rate of the user 205 may also be factored in to determine t-max and further refine the gain term.

At step 805 of the flow diagram 800, the ambulatory medical device 220 may receive, from a sensor, one or more biomarker measurements in the blood of the user 205. Examples of biomarkers include blood glucose levels and ketone levels in the user 205. An example of a sensor that may collect these biomarkers is the biochemical sensor 165, as shown in FIG. 1.

At step 810 of the flow diagram 800, the ambulatory medical device 220 may determine a response to one or more medicaments based on the one or more biomarker measurements. An example of a response may be a change in blood glucose level following the administration of one or more medicaments.

Examples of the one or more medicaments include insulin and glucagon. For example, the processor 120 may determine that a blood glucose level responded to an increase in glucagon or insulin administration.

At step 815 of the flow diagram 800, the ambulatory medical device 220 may adjust the gain term based on the response to one or more medicaments. The gain term is configured to control the amount of medicament administered from the ambulatory medical device 220. For example, if biomarker measurements indicate that the user 205's blood glucose level remains above a threshold over a period of time, the gain term may be adjusted upward. Likewise, if the blood glucose level remains below a threshold over a period of time following the previous administration of insulin, the gain term may be adjusted downward.

Other statistics may also be considered, including standard deviation. For example, if the standard deviation of blood glucose levels over a period of time is high, the gain term may be increased. Conversely, if the standard deviation over a period of time is low, the gain term may be decreased.

At step 820 of the flow diagram 800, the ambulatory medical device 220 may transmit an instruction to the pump assembly 135 to deliver the determined amount of medicament to the user 205. The instruction may be transmitted from the controller 105 to the pump assembly 135 via a wired or wireless connection. In some embodiments, the pump assembly 135 may operate a pump that infuses the medicament through the flexible tubing 330 into a transdermal syringe 215 for delivery to the user 205.

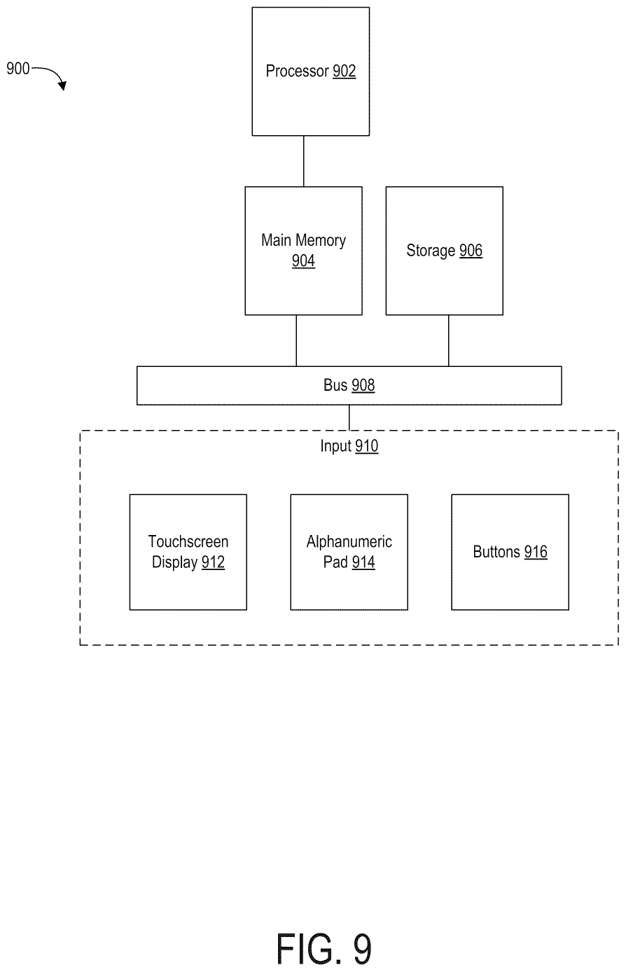

Referring to FIG. 9, FIG. 9 is a block diagram illustrating the computer system 900 that may be implemented in the various embodiments in the described subject matter. The computer system 900 includes a processor 902, main memory 904, storage 906, a bus 908, and input 910. The processor 902 may be one or more processors. The processor 902 executes instructions that are communicated to the processor through the main memory 904. The main memory 904 feeds instructions to the processor 902. The main memory 904 is also connected to the bus 908. The main memory 904 may communicate with the other components of the computer system through the bus 908. Instructions for the computer system 900 are transmitted to the main memory 904 through the bus 908. Those instructions may be executed by the processor 902. Executed instructions may be passed back to the main memory 904 to be disseminated to other components of the computer system 900. The storage 906 may hold large amounts of data and retain that data while the computer system 900 is unpowered. The storage 906 is connected to the bus 908 and can communicate data that the storage holds to the main memory 904 through the bus 908.

The processor 902 may be any type of general purpose processor including, but not limited to a central processing unit (“CPU”), a graphics processing unit (“GPU”), a complex programmable logic device (“CPLD”), a field programmable gate array (“FPGA”), or an application-specific integrated circuit (“ASIC”). One embodiment of the computer system 900 in the ambulatory medical device features a CPU as the processor 902. However, embodiments may be envisioned for the computer system of the ambulatory medical device that incorporate other types of processors 902.

The main memory 904 can be any type of main memory that can communicate instructions to the processor 902 and receive executed instructions from the processor 902. Types of main memory 904 include but are not limited to random access memory (“RAM”) and read only memory (“ROM”). In one embodiment, the computer system 900 incorporates RAM as the form of main memory 904 to communicate instructions to the processor 902 and receive executed instructions from the processor 902. Other embodiments may be envisioned that incorporate other types of main memory 904 in the computer system 900.

The storage 906 can be any type of computer storage that can receive data, store data, and transmit data to the main memory 904 via the bus 908. Types of storage 906 that can be used in the computer system 900 include, but are not limited to, magnetic disk memory, optical disk memory, and flash memory. In one embodiment, flash memory is used as the storage 906 in the computer system 900 of the ambulatory medical device. Other embodiments that use other types of storage 906 for the computer system 900 may be envisioned.

The bus 908 connects the internal components of the computer system 900. The bus 908 may include a multitude of wires that are connected to the components of the computer system 900. The wires of the bus 908 may differ based on the components of the computer system 900 that the bus 908 connects to. In various embodiments, the bus 908 connects the processor 902 to the main memory 904. In various embodiments, the processor 902 is directly connected to the main memory 904.

The input 910 of the computer system 900 includes a touchscreen display 912, an alphanumeric pad 914, and buttons 916. The touchscreen display 912 both produces output and accepts input. The bus 908 may be coupled to the touchscreen display 912 to produce visual output. The touchscreen display 912 may also accept input via capacitive touch, resistive touch, or other touch technology. The input surface of the touchscreen display 912 can register the position of touches on the surface. Some types of touchscreen display 912 can register multiple touches at once. The alphanumeric pad 914 includes a multitude of keys with numerical, alphabetical, and symbol characters. Signals from the alphanumeric pad 914 are communicated by the bus 908 to the main memory 904. Keys of the alphanumeric pad may be capacitive or mechanical. In some embodiments, the alphanumeric pad 914 is displayed on the touchscreen display 912. Buttons 916, such as a meal announcement button, may be capacitive, mechanical, or other single input buttons. or other single input buttons.

A method for administering medicament from a medical device includes receiving, from a sensor, one or more amounts of biomarkers in blood of a user, determining a response to one or more medicaments responsive to the one or more amounts of biomarkers, adjusting a gain-term based on the response to the one or more medicaments, the gain-term configured to control an amount of medicament that is administered from an ambulatory medical device, and transmitting an instruction to the ambulatory medical device to deliver the amount of medicament to the user. The response is a glucose concentration over a period of time, and the gain term is adjusted responsive to the glucose concentration being below or above a target threshold for greater or less than a time condition. The gain term is adjusted based on a mean glucose concentration and is further adjusted based on a standard deviation of the glucose concentration. Determining a basal rate for one or more biomarkers in the user is included, and adjusting the gain-term is further based on the basal rate.

A system for administering medicament to a user includes an ambulatory medication administration device, the ambulatory medication administration device including a reservoir configured to store an amount of medicament, a pump configured to dispense the medicament from the reservoir to a user carrying the ambulatory medication administration device, a sensor configured to collect a biomarker concentration in the user, and a controller. The controller is configured to receive, from the sensor, the biomarker concentration, determine a response to a medicament responsive to the biomarker concentration, adjust a gain-term based on the response to the one or more medicaments, the gain-term configured to control an amount of medicament that is administered from an ambulatory medical device, and transmit, responsive to the biomarker concentration, an instruction to the pump to dispense the medicament. The response is a glucose concentration over a period of time, and the gain term is adjusted responsive to the glucose concentration being below or above a target threshold for greater or less than a time condition. The gain term is adjusted based on a mean glucose concentration and is further adjusted based on a standard deviation of the glucose concentration. Determining a basal rate for one or more biomarkers in the user is included, and adjusting the gain-term is further based on the basal rate.

An ambulatory medical device includes a reservoir configured to store an amount of medicament, a pump configured to dispense the medicament from the reservoir to a user carrying the ambulatory medication administration device, a sensor configured to collect a biomarker concentration in the user, and a controller. The controller is configured to receive, from the sensor, the biomarker concentration, determine a response to a medicament responsive to the biomarker concentration, adjust a gain-term based on the response to the one or more medicaments, the gain-term configured to control an amount of medicament that is administered from an ambulatory medical device, and transmit, responsive to the biomarker concentration, an instruction to the pump to dispense the medicament. The response is a glucose concentration over a period of time, and the gain term is adjusted responsive to the glucose concentration being below or above a target threshold for greater or less than a time condition. The gain term is adjusted based on a mean glucose concentration and is further adjusted based on a standard deviation of the glucose concentration.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A method for administering medicament from a medical device, the method comprising:
receiving, from a sensor, one or more amounts of biomarkers in blood of a user;
determining a response to one or more medicaments responsive to the one or more amounts of biomarkers;
adjusting a gain term based on the response to the one or more medicaments, the gain-term configured to control an amount of medicament that is administered from an ambulatory medical device; and
transmitting an instruction to the ambulatory medical device to deliver the amount of medicament to the user.

2. The method of claim 1, wherein the response is a glucose concentration over a period of time.

3. The method of claim 2, wherein the gain term is adjusted responsive to the glucose concentration being below a target threshold for greater or less than a time condition.

4. The method of claim 2, wherein the gain term is adjusted responsive to the glucose concentration being above a target threshold for greater or less than a time condition.

5. The method of claim 2, wherein the gain term is adjusted based on a mean glucose concentration.

6. The method of claim 5, wherein the gain term is further adjusted based on a standard deviation of the glucose concentration.

7. The method of claim 1, further comprising determining a basal rate for one or more biomarkers in the user; and
wherein adjusting the gain term is further based on the basal rate.

8. A system for administering medicament to a user, the system comprising:
an ambulatory medication administration device, the ambulatory medication administration comprising:
a reservoir configured to store an amount of medicament;
a pump configured to dispense the medicament from the reservoir to a user carrying the ambulatory medication administration device;
a sensor configured to collect a biomarker concentration in the user; and
a controller, the controller configured to:
receive, from the sensor, the biomarker concentration;
determine a response to a medicament responsive to the biomarker concentration;
adjust a gain term based on the response to the medicament, the gain term configured to control an amount of medicament that is administered from an ambulatory medical device; and
transmit, responsive to the biomarker concentration, an instruction to the pump to dispense the medicament.

9. The system of claim 8, wherein the response is a glucose concentration over a period of time.

10. The system of claim 9, wherein the gain term is adjusted responsive to the glucose concentration being below a target threshold for greater or less than a time condition.

11. The system of claim 9, wherein the gain term is adjusted responsive to the glucose concentration being above a target threshold for greater or less than a time condition.

12. The system of claim 9, wherein the gain term is adjusted based on a mean glucose concentration.

13. The system of claim 12, wherein the gain term is further adjusted based on a standard deviation of the glucose concentration.

14. The system of claim 8, further comprising determining a basal rate for one or more biomarkers in the user; and
wherein adjusting the gain term is further based on the basal rate.

15. An ambulatory medical device, the ambulatory medical device comprising:
a reservoir configured to store an amount of medicament;
a pump configured to dispense the medicament from the reservoir to a user carrying the ambulatory medical administration device;
a sensor configured to collect a biomarker concentration in the user; and
a controller, the controller configured to:
receive, from the sensor, the biomarker concentration;
determine a response to a medicament responsive to the biomarker concentration;
adjust a gain term based on the response to the medicament, the gain term configured to control an amount of medicament that is administered from an ambulatory medical device; and
transmit, responsive to the biomarker concentration, an instruction to the pump to dispense the medicament.

16. The ambulatory medical device of claim 15, wherein the response is a glucose concentration over a period of time.

17. The ambulatory medical device of claim 16, wherein the gain term is adjusted responsive to the glucose concentration being below a target threshold for greater or less than a time condition.

18. The ambulatory medical device of claim 9, wherein the gain term is adjusted responsive to the glucose concentration being above a target threshold for greater or less than a time condition.

19. The ambulatory medical device of claim 9, wherein the gain term is adjusted based on a mean glucose concentration.

20. The ambulatory medical device of claim 15, further comprising determining a basal rate for one or more biomarkers in the user; and
wherein adjusting the gain-term is further based on the basal rate.
